(19) United States
(12) Patent Application Publication
(10) Pub. No.: US 2015/0374844 A1
(43) Pub. Date: Dec. 31, 2015

(54) NOVEL PH -SWITCHABLE PEPTIDES FOR MEMBRANE INSERTION AND PORE FORMATION

(71) Applicants: UNIVERSITY OF PENNSYLVANIA, Philadelphia, PA (US); SIRNA THERAPEUTICS, INC., Cambridge, MA (US)

(72) Inventors: William DEGRADO, Philadelphia, PA (US); David M. TELLERS, West Point, PA (US); Gevorg GRIGORYAN, Philadelphia, PA (US); Vasant JADHAV, West Point, PA (US); Yao ZHANG, Philadelphia, PA (US)

(21) Appl. No.: 14/768,207

(22) PCT Filed: Feb. 10, 2014
(86) PCT No.: PCT/US14/15476

§ 371 Date: Aug. 14, 2015

Related U.S. Application Data
(60) Provisional application No. 61/765,097, filed on Feb. 15, 2013.

(51) Int. Cl. A61K 47/48; A61K 31/713; C07K 14/00

(52) U.S. Cl. CPC A61K 47/48246; C07K 14/001; A61K 31/713

(57) ABSTRACT

Disclosed herein is a pH-switchable pore formation (PSPF) peptide comprising one or more amino acids in peptide sequence whose charge state and hydrophobicity are pH-dependent, wherein the peptide can bind to a biological membrane upon contact and form pores on the membrane at pH of less than about 7, and wherein the peptide forms substantially no pores on the biological membrane at pH of greater than about 7. Also disclosed is a modular composition comprising: a) one or more PSPF peptides, which may be the same or different; b) a single stranded or double stranded oligonucleotide; and c) one or more linkers, which may be the same or different.

### BACKGROUND OF THE INVENTION

As therapeutic potentials for macromolecules, like peptides and proteins, are increasingly characterized, efforts to develop a variety of intracellular drug delivery systems as viral vector, lipoplexes, nanoparticles and amphiphilic peptides have been made. The ability to introduce targeted substances into a cell's interior would greatly enhance the ability to interface with cellular processes, but various challenges such as delivery efficiency, toxicity and controllability remain to be overcome.

Though for a small class of molecules cellular uptake can be spontaneous, the general task, known as the delivery problem, is largely unsolved. This is because biological membranes serve as effective barriers that prevent most substances from freely flowing into and out of cells and between organelles.

There is a continuing need to develop means to deliver these macromolecules across the hydrophobic barrier of membrane into the cytosolic environment where these agents carry out the expected functions.

### SUMMARY OF THE INVENTION

Disclosed herein are a series of pH-switchable pore formation (PSPF) peptides as potential delivery agents. In one embodiment, a PSPF peptide comprises one or more amino acids in peptide sequence whose charge state and hydrophobicity are pH-dependent, wherein the PSPF peptide can bind to a biological membrane upon contact and form pores on the membrane at pH of less than about 7, and wherein the PSPF peptide forms substantially no pores on the biological membrane at pH of greater than about 7.

### BRIEF DESCRIPTION OF THE DRAWINGS

FIG. 1. Desired free energy diagram of a PSPF peptide as a function of pH. Lowering pH destabilizes water-soluble bundle state and stabilizes first membrane-associated monomeric state and then, in a concentration-dependent manner, the membrane-inserted channel state.

FIG. 2. Design concept using one of the disclosed sequences (PSPF-DKG).

FIG. 3. Correlation between ATP-release by PSPF peptides and the degree of lipid engagement as assessed by the fractional change of Trp-fluorescence signal upon addition of 200 μM lipid vesicles.

FIG. 4. Size exclusion chromatography of PSPF-EKG and PSPF-DKG at each pH.

FIG. 5. AUC sedimentation equilibrium of PSPF-EKG at pH 5.5 (A) and 7.4 (C).

FIG. 6. AUC sedimentation equilibrium of PSPF-DKG at pH 5.5 and 7.4.

FIG. 7. Circular dichroism of PSPF-EKG suggests an alpha-helical secondary structure at both pHs.

FIG. 8. Thermal denaturation of PSPF-EKG at pH 7.4 (A) and 5.5 (B). The data are fit to the Gibbs-Helmholtz Equation.

FIG. 9. The single-species fitting of AUC sedimentation in detergent micelles for PSPF-EKG at pH 7.4 (A) and 5.5 (C). Species weight fraction of PSPF-EKG at pH 7.4 (C) and pH 5.5 (D) as the data were globally fit to a monomer-trimer equilibrium as an example.

FIG. 10. ATR-IR of PSPF-EKG in phospholipids (POPC) bilayers.

FIG. 11. Model of PSPF-EKG membrane insertion and pore formation upon pH decrease.

### DETAILED DESCRIPTION OF THE INVENTION

Disclosed herein are a series of pH-switchable pore formation (PSPF) peptides as therapeutic agents and/or delivery vehicles for therapeutic agents.

Therapeutic macromolecules such as peptides and proteins are easily cleared from the bloodstream and require assistance for intracellular delivery in order to reach their intended targets and achieve the desirable therapeutic effects. Decades of research effort have been devoted to develop delivery agents with high efficiency and low toxicity and the results have not been satisfactory.

Viral vectors have been extensively studied for gene therapy. Viral vector based gene therapy has demonstrated promising results, but this potential life-saving delivery technique can also be risky. The death of a patient in a trial suggests that viral vectors might also induce undesirable gene insertion and this potential danger is currently uncontrollable.

Most non-viral carriers are synthetic chemical conjugates. Active pharmaceutical ingredients are usually linked or enclosed into a vehicle and delivered into the cell via endocytosis or membrane fusion, or via a yet to be determined mechanism. These vehicles are typically designed as liposomes/lipoplexes, cationic macromolecules polymer, polypeptide, protein, amphiphilic polymer/polypeptide, nanoparticles and cell penetrated peptides (CPP). Native sequences such as fusogenic peptides from viral fusion protein have also been manipulated as a cargo carrier to cross the barrier of cell membranes. A number of these approaches have also entered clinical trials, but most of them reached a bottleneck due to high toxicity or lack of manipulability.

Disclosed herein are a series of pH-switchable pore forming peptides as therapeutic agents and/or vehicles for intracellular (lysosomal) drug delivery.

To allow flux of desired target, organisms depend on membrane-inserted protein channels and transporters. Thus a potential solution to the delivery problem is via engineering of custom channels or transporters.

In nature, a common feature of these carrier proteins is their controllability. A channel or transporter responsible for the flux of an important molecule can generally be activated or inactivated by the cell as needed. For example, channel-forming toxin peptides, found in each of the three domains of life, generally become active after a proteolytic cleavage event. This is also a desirable feature in engineered carrier proteins as controlled delivery could lead to targeted delivery in pharmaceutical applications.

PSPF peptides disclosed herein can bind to biological membranes and form pores only at low pH, for example less than about 7, but are minimally interactive at high pH, for example greater than about 7. The pores can serve as channels for transport of appropriately-sized target, while the pH switch provides a convenient manner in which to control the activity. Further, because of the lower pH environment in the endosome, the uptake of such peptides by endocytosis could allow endosomal escape of material present in the extracellular environment into the cell.

In one embodiment, a PSPF peptide comprises one or more amino acids in peptide sequence whose charge state and hydrophobicity are pH-dependent, wherein the peptide can bind to a biological membrane upon contact and form pores on the membrane at pH of less than about 7, and wherein the peptide forms substantially no pores on biological membranes at pH of greater than about 7.

In another embodiment, the PSPF peptide can bind to a biological membrane upon contact and form pores on the membrane at pH of less than 6.5, and wherein the peptide forms substantially no pores on biological membranes at pH of greater than 7.0.

In another embodiment, the PSPF peptide can bind to a biological membrane upon contact and form pores on the membrane at pH of about 5.5, and wherein the peptide forms substantially no pores on biological membranes at pH of about 7.4.

In one embodiment, the PSPF peptide is water soluble at pH of greater than about 7. In another embodiment, the PSPF peptide is water soluble at pH of about 7.4.

In another embodiment, the amino acid in the PSPF peptide is selected from the group consisting of Asp, Glu and His.

In another embodiment, the pores formed on the membrane can serve as channels for transport of appropriately-sized target.

In another embodiment, PSPF conjugated materials present in the extracellular environment can be taken up by endocytosis followed by PSPF mediated release of conjugated material from the endolysosomal compartment to the cytosol.

In another embodiment, the PSPF peptide is selected from peptides of SEQ. ID No. 1-24.

In another embodiment, the peptide is selected from peptides of SEQ. ID No. 4, 8 and 12.

Also disclosed herein is a modular composition comprising: a) one or more PSPF peptides disclosed herein, which may be the same or different; b) a single stranded or double stranded oligonucleotide; c) optionally one or more linkers, which may be the same or different; d) optionally one or more targeting ligands, which may be the same or different; e) optionally one or more other peptides; and f) optionally one or more lipids, which may be the same or different.

In one embodiment, a modular composition comprises: a) one or more PSPF peptides, which may be the same or different; b) a single stranded or double stranded oligonucleotide; and c) one or more linkers, which may be the same or different. In one embodiment, the modular composition further comprises d) one or more targeting ligands, which may be the same or different.

In one embodiment, each ligand is independently selected from the group consisting of D-galactose, N-acetyl-D-galactosamine (GalNAc), GalNAc2, and GalNAc3, GalNAc4, cholesterol, folate, and analogs and derivatives thereof.

In one embodiment, the oligonucleotide of the modular composition above is siRNA. In another embodiment, the siRNA is single stranded. In another embodiment, the siRNA is double stranded.

In one embodiment of the modular composition above, the siRNA is double stranded; and each peptide is independently selected from peptides of SEQ. ID 1-24.

In one embodiment, a modular composition comprises a) 1-4 PSPF peptides independently selected from SEQ ID No. 1-24; b) a double stranded siRNA; c) 1-4 linkers independently selected from Table 8, which may be the same or different; and d) 1-4 GalNAc ligands, which may be the same or different; and wherein the GalNAc ligands and/or the peptides are attached to the siRNA optionally via linkers.

In one embodiment, the GalNAc ligands and the peptides are attached to the same strand of the siRNA via linkers.

In one embodiment, a modular composition comprises: a) one or more PSPF peptides, which may be the same or different; b) a single stranded or double stranded oligonucleotide; c) one or more linkers, which may be the same or different; d) optionally one or more targeting ligands, which may be the same or different; e) optionally one or more other peptides; and f) optionally one or more lipids, which may be the same or different.

In another embodiment, a modular composition comprises: a) one or more PSPF peptides, which may be the same or different; b) a single stranded or double stranded oligonucleotide; c) one or more linkers, which may be the same or different; d) one or more targeting ligands, which may be the same or different; e) optionally one or more other peptides; and f) optionally one or more lipids, which may be the same or different.

In yet another embodiment, a modular composition comprises: a) one or more PSPF peptides, which may be the same or different; b) a single stranded or double stranded oligonucleotide; c) one or more linkers, which may be the same or different; d) one or more targeting ligands, which may be the same or different; e) one or more other peptides; and f) one or more lipids, which may be the same or different.

In one embodiment, a pharmaceutical composition comprises a PSPF peptide disclosed herein and a pharmaceutically acceptable excipient.

In one embodiment, a pharmaceutical composition comprises a modular composition disclosed herein and a pharmaceutically acceptable excipient.

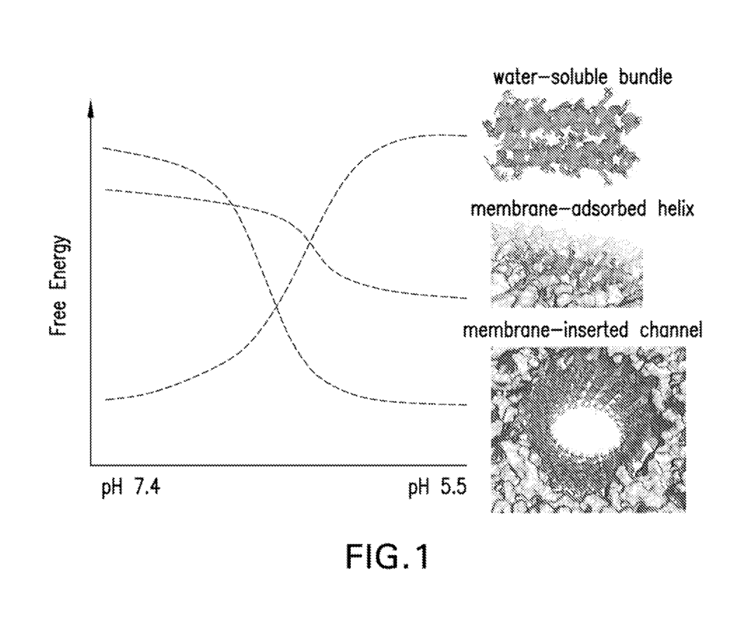

To realize the pH-switchable behavior described above, three thermodynamic states are considered to arrive at the desired PSPF peptides, as shown in FIG. 1, which shows the desired free energy diagram of the peptide as a function of pH. At high pH, for example greater than about 7, or more specifically at about 7.4, the peptide should be “stored” in a water-soluble form that does not interact with the membrane. A good way to encode this is to assure the formation of a stable water-soluble helical bundle at high pH. Lowering of pH, for example to less than 6.5, or more specifically to about 5.5, should destabilize this state, allowing peptide monomers to interact with the membrane. This can be achieved either by a surface-adsorbed form, in which helical monomers are engaged with the membrane surface, or a fully inserted state capable of forming a channel. Because insertion and channel formation are thermodynamically linked, the relative stability of the inserted versus surface-adsorbed states will have a concentration dependence, with higher peptide concentrations favoring insertion and channel formation.

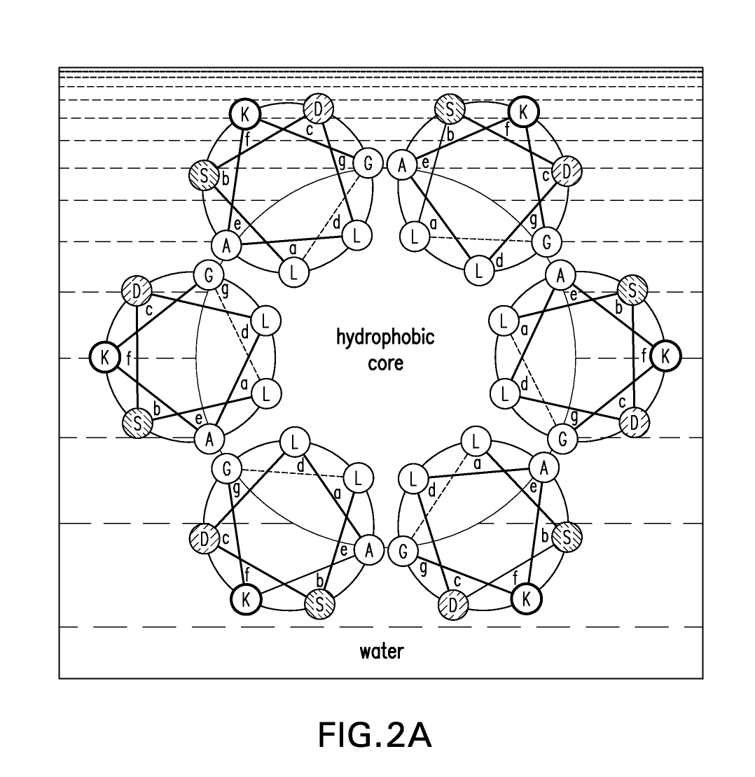

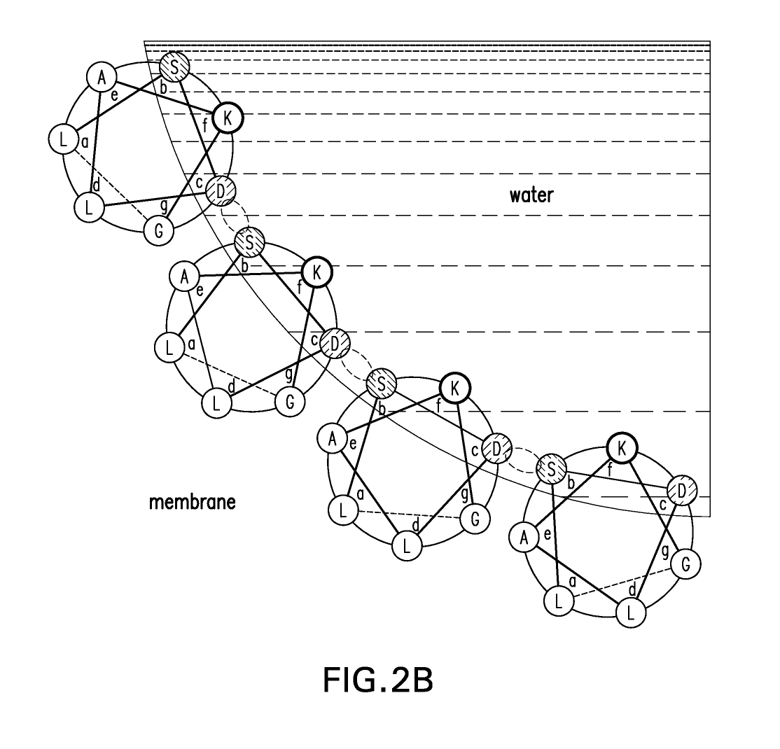

In one embodiment, the PSPF peptides contain amphipatic helices, which consist of hydrophobic, non-polar residues on one side of the helical cylinder and hydrophilic and polar residues on the other side, resulting in a hydrophobic moment. In this way, they aggregate with other hydrophobe surfaces and serve for example as pores or channels in the cell membrane. Some amphipatic helices are arranged as intertwined helices that are termed a coiled-coils or super-helices. Generally, the sequence of an alpha helix that participates in a coiled-coil region will display a periodicity with a repeated unit of length 7 amino acids, which is called a “heptad” repeat, as illustrated in FIG. 2. Denote those 7 positions by letters “a” through “g”, then position “a” and “d” are hydrophobic and define an apolar stripe, while there exist electrostatic or other favorable interactions between residues at positions “e” and “g”.

To minimize membrane association at high pH, the water-soluble bundle should be very stable and its exterior should interact more favorably with water than the membrane at these conditions. The most hydrophobic and potentially membrane-interacting region of the peptide is buried in the core in this state. At low pH, both of these factors ideally need to be reversed—the stability of the water-soluble bundle should decrease, producing a population of dissociated monomers poised to interact with the membrane, while the hydrophobicity of the peptide (and thus its preference to interact with the membrane) should increase.

This pH modulation of stability and hydrophobicity can be achieved by including amino acids in the peptide sequence whose charge state and hydrophobicity are pH-dependent, such as Asp, Glu and His, and considering the stability of the water-soluble coiled coil-like bundle. In addition, the specific inter-residue interactions of the membrane-inserted pore are also considered in selecting the desired sequence, as a specific pore-forming state at low pH, rather than simply ensuring membrane insertion.

For example, peptides that simply insert into membranes or those that insert and form indiscriminately large pores or even cause lysis are abundant in nature, but would constitute unsuccessful endpoints either because of lack of pore formation or potential toxicity. Thus, to arrive at a desired peptide, both the use of pH-switchable residues and consideration of inter-residue contacts and stabilities of both the water-soluble as well as membrane-inserted pore states are needed.

In one embodiment, a PSPF peptide disclosed herein associates with the membrane in a pH dependent manner and capable of pH dependent pore formation.

In one embodiment, a PSPF peptide is a water-soluble peptide that associates into a stable coiled-coil bundle at high-to-neutral pH, while preferring a membrane-inserted channel state at low pH. This means that upon pH decrease, the nonpolar residues facing inward in the soluble bundle, should invert and face the lipid phase in the membrane-inserted channel, as shown in FIG. 2.

Since canonical coiled coils have only seven environmentally distinct positions, referred to as the heptad and designated with letters “a” though “g” (FIG. 2A), each site of “a” through “g” plays two roles—stabilizing the water-soluble, “hydrophobic-inside” state at high pH and the membrane channel, “hydrophobic-outside” state at low pH. To impart stability on the water-soluble bundle, the canonical Leu-zipper coiled-coil motif was chosen, meaning that coiled-coil positions “a” and “d” were set to Leu. These same residues face the lipid phase in the membrane channel state, and Leu residues are ideal for this task as well (FIG. 2B). The solvent-exposed “b”, “c”, and “f” positions in the water-soluble bundle should be polar to impart solubility and fold specificity, and these can also be used to modulate bundle stability through their innate helix propensities.

In the membrane-channel state, these positions are also water-facing, as they point into the center of the channel, so their polar nature is appropriate here as well. However, unlike in the water-soluble state, “b” and “c” positions are also located at the inter-helical interface of the channel. Thus, the importance of these positions goes beyond their physico-chemical character and includes potential interactions stabilizing specific interfacial conformations of channel helices. The inter-helical geometry in the channel state is important as it ultimately defines the shape and even size of the entire channel.

FIG. 2 illustrates the design concept using one of the designed sequences (PSPF-DKG). Hydrophobic residues are either lining the bundle at the core in the water-soluble state (A), or are facing the lipid membrane in the membrane channel state (B). Dotted circles illustrate potential hydrogen bonding in the channel state. Heptad positions in both panels are labeled according to the water-soluble state.

In one embodiment, exemplary amino acid choices at each position are shown in Table 1.

TABLE 1
Exemplary Amino Acid Choices

| Position | Function in water, | Position | Function in membrane, | Exemplary |
|---|---|---|---|---|
| in water | high pH | in membrane | low pH | Amino acid |
| a | Helical bundle | c | Membrane-facing | Leu |
| | hydrophobic core | | | |
| b | Solvent-exposed, | d | Small residue for helical | Ser |
| | imparts solubility | | interface, potential inter-helical | |
| | | | hydrogen bonding | |
| c | Solvent-exposed, | e | Trigger residue, changes | Asp, Glu, His |
| | imparts solubility | | protonation state/hydrophobicity | |
| | | | at low pH. Potential inter-helical | |
| | | | hydrogen bonding. | |
| d | Helical bundle | f | Membrane-facing | Leu |
| | hydrophobic core | | | |
| e | Modulation of | g | Small residue for helical interface | Ala |
| | helical propensity | | | |
| f | Solvent-exposed, | a | Solvent-exposed in channel state | Lys, Gln |
| | imparts solubility | | (inner channel lining). Imparts | |
| | | | folds specificity by encoding | |
| | | | helical orientation preference | |
| g | Modulation of | b | Small residue for helical interface | Ala, Gly |
| | helical propensity. | | | |

At the “f” position in water, polar amino acids Lys and Gln can be used. Any other natural or unnatural amino acids that maintain water-solubility of the protein (e.g.; A, C, D, E, G, H, K, N, Orn, Q, R, S, T, Y, alpha-amino-isobutyric acid), can also be used.

At position “b” in water, Ser can be used because of its polar nature, as well as its high preponderance in closely-packing helix-helix interfaces in TM proteins. Additional small, polar natural and unnatural amino acids such as Ala, Thr, Cys alpha-amino-isobutyric acid, alpha-amino-butyric acid, and Met can also be used.

At positions “a” and “d” in water, Leu, or a similar non-polar natural or unnatural amino acid such as Ala, Val, Phe, norleucine, alpha-amino-isobutyric acid, alpha-amino-butyric acid, Met and Ile can be used.

The “c” position in water was chosen as the pH-sensing switch Amino acids Glu and Asp can be used at this position as their protonation state is dependent on pH, causing them to be more protonated, less charged and thus more hydrophobic at lower pH. Although the pKa of the carboxylic side-chain groups of Glu and Asp in water are around 4.0, somewhat lower than the typical endosomal pH of ˜5.5, significant shifting in protonated populations would still be expected relative to neutral pH, and the collective effect of having multiple closely-spaced acidic groups on one face of a helix will likely increase the effective pKa of the side-chains. An additional significance of Glu and Asp residues is their potential ability to participate in inter-helical hydrogen bonding (FIG. 2B), thus further dialing in a specific, closely packed inter-helical geometry in the membrane-channel state. Note that additional longer chain natural and unnatural amino acids with similar pH responsive properties such as His, and longer chain analogues of Glu (i.e., with side chains consisting of (CH₂)ₙ—COOH where n=3-6) can also be used.

As a way of testing the importance of the pH switch residue, using amino acid His at the “c” position was also considered. The side-chain of His titrates at pH ˜6.1, but it is more charged at acidic pH than at neutral pH. Because of this reversed pH sensitivity compared to Asp and Glu, His provides a convenient point of reference.

Positions “e” and “g” in water are located along the helix-helix interface in both the water-soluble and the membrane-channel states. Because the primary driver of the water-soluble bundle stability is the canonical Leu-zipper motif, small hydrophobic residues at “e” and “g” were chosen with the primary purpose of stabilizing a closely-packed TM helical interface. Additional non-polar natural and unnatural amino acids can be used here as well. Examples include Ala, Gly, Ser, Cys, alpha-amino-isobutyric acid, alpha-amino-butyric acid, and Thr.

In one embodiment, a PSPF peptide is selected from peptides of Seq. ID 1-24 as shown in Table 2. Note that for the first heptad, the position “c” can be substituted with a tryptophan which is used for spectrophotometric purposes. In addition, either termini can be substituted with additional moieties to allow for conjugation to an oligonucleotide.

TABLE 2
The Sequence of PSPF Peptides

| Peptide | Seq. ID | Peptide Sequence |
|---|---|---|
| Heptad in membrane | | cdefgab cdefgab cdefgab cdefgab |
| PSPF-DQA | 1 | WSDLAQA LSDLAQA LSDLAQA LSDLAQA |
| PSPF-DQG | 2 | WSDLAQG LSDLAQG LSDLAQG LSDLAQG |
| PSPF-DKA | 3 | WSDLAKA LSDLAKA LSDLAKA LSDLAKA |
| PSPF-DKG | 4 | WSDLAKG LSDLAKG LSDLAKG LSDLAKG |
| PSPF-EQA | 5 | WSELAQA LSELAQA LSELAQA LSELAQA |
| PSPF-EQG | 6 | WSELAQG LSELAQG LSELAQG LSELAQG |
| PSPF-EKA | 7 | WSELAKA LSELAKA LSELAKA LSELAKA |
| PSPF-EKG | 8 | WSELAKG LSELAKG LSELAKG LSELAKG |
| PSPF-HQA | 9 | WSHLAQA LSHLAQA LSHLAQA LSHLAQA |
| PSPF-HQG | 10 | WSHLAQG LSHLAQG LSHLAQG LSHLAQG |
| PSPF-HKA | 11 | WSHLAKA LSHLAKA LSHLAKA LSHLAKA |
| PSPF-HKG | 12 | WSHLAKG LSHLAKG LSHLAKG LSHLAKG |
| PSPF-DQA-GGC | 13 | WSDLAQA LSDLAQA LSDLAQA LSDLAQAGGC |
| PSPF-DQG-GGC | 14 | WSDLAQG LSDLAQG LSDLAQG LSDLAQGGGC |
| PSPF-DKA-GGC | 15 | WSDLAKA LSDLAKA LSDLAKA LSDLAKAGGC |
| PSPF-DKG-GGC | 16 | WSDLAKG LSDLAKG LSDLAKG LSDLAKGGGC |
| PSPF-EQA-GGC | 17 | WSELAQA LSELAQA LSELAQA LSELAQAGGC |
| PSPF-EQG-GGC | 18 | WSELAQG LSELAQG LSELAQG LSELAQGGGC |
| PSPF-EKA-GGC | 19 | WSELAKA LSELAKA LSELAKA LSELAKAGGC |
| PSPF-EKG-GGC | 20 | WSELAKG LSELAKG LSELAKG LSELAKGGGC |
| PSPF-HQA-GGC | 21 | WSHLAQA LSHLAQA LSHLAQA LSHLAQAGGC |
| PSPF-HQG-GGC | 22 | WSHLAQG LSHLAQG LSHLAQG LSHLAQGGGC |
| PSPF-HKA-GGC | 23 | WSHLAKA LSHLAKA LSHLAKA LSHLAKAGGC |
| PSPF-HKG-GGC | 24 | WSHLAKG LSHLAKG LSHLAKG LSHLAKGGGC |
| Heptad in Water | | abcdefg abcdefg abcdefg abcdefg |

As used herein, the three-letter and single-letter codes for amino acids are well known in the art and listed in Table 3.

TABLE 3
Three-letter and Single-letter Codes for Amino Acids

| Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Also disclosed herein is a method of delivering an oligonucleotide to a cell. In one embodiment, the method includes (a) providing or obtaining a modular composition comprising one or more PSPF peptides disclosed herein; (b) contacting a cell with the modular composition; and (c) allowing the cell to internalize the modular composition.

The method can be performed in vitro, ex vivo or in vivo, e.g., to treat a subject identified as being in need of an oligonucleotide. A subject in need of said oligonucleotide is a subject, e.g., a human, in need of having the expression of a gene or genes, e.g., a gene related to a disorder, downregulated or silenced.

In one embodiment, the invention provides a method for inhibiting the expression of one or more genes. The method comprises contacting one or more cells with an effective amount of a PSPF peptide or a modular composition of the invention, wherein the effective amount is an amount that suppresses the expression of the one or more genes. The method can be performed in vitro, ex vivo or in vivo.

The methods and compositions of the invention, e.g., the modular composition described herein, can be used with any oligonucleotides known in the art. In addition, the methods and compositions of the invention can be used for the treatment of any disease or disorder known in the art, and for the treatment of any subject, e.g., any animal, any mammal, such as any human. One of ordinary skill in the art will also recognize that the methods and compositions of the invention may be used for the treatment of any disease that would benefit from downregulating or silencing a gene or genes.

The methods and compositions of the invention, e.g., the modular composition described herein, may be used with any dosage and/or formulation described herein, or any dosage or formulation known in the art. In addition to the routes of administration described herein, a person skilled in the art will also appreciate that other routes of administration may be used to administer the modular composition of the invention.

### Oligonucleotide

An “oligonucleotide” as used herein, is a double stranded or single stranded, unmodified or modified RNA or DNA. Examples of modified RNAs include those which have greater resistance to nuclease degradation than do unmodified RNAs. Further examples include those which have a 2′ sugar modification, a base modification, a modification in a single strand overhang, for example a 3′ single strand overhang, or, particularly if single stranded, a 5′ modification which includes one or more phosphate groups or one or more analogs of a phosphate group. Examples and a further description of oligonucleotides can be found in WO2009/126933, which is hereby incorporated by reference.

In one embodiment, an oligonucleotide is an antisense, miRNA, peptide nucleic acid (PNA), poly-morpholino (PMO) or siRNA. The preferred oligonucleotide is an siRNA. Another preferred oligonuleotide is the passenger strand of an siRNA. Another preferred oligonucleotide is the guide strand of an siRNA.

siRNA

siRNA directs the sequence-specific silencing of mRNA through a process known as RNA interference (RNAi). The process occurs in a wide variety of organisms, including mammals and other vertebrates. Methods for preparing and administering siRNA and their use for specifically inactivating gene function are known. siRNA includes modified and unmodified siRNA. Examples and a further description of siRNA can be found in WO2009/126933, which is hereby incorporated by reference.

A number of exemplary routes of delivery are known that can be used to administer siRNA to a subject. In addition, siRNA can be formulated according to any exemplary method known in the art. Examples and a further description of siRNA formulation and administration can be found in WO2009/126933, which is hereby incorporated by reference.

The phrases “short interfering nucleic acid”, “siNA”, “short interfering RNA”, “siRNA”, “short interfering nucleic acid molecule”, “oligonucleotide”, “short interfering oligonucleotide molecule”, or “chemically modified short interfering nucleic acid molecule” refer to any nucleic acid molecule capable of inhibiting or down regulating gene expression or viral replication by mediating RNA interference (“RNAi”) or gene silencing in a sequence-specific manner. These terms can refer to both individual nucleic acid molecules, a plurality of such nucleic acid molecules, or pools of such nucleic acid molecules. The siNA can be a double-stranded nucleic acid molecule comprising self-complementary sense and antisense strands, wherein the antisense strand comprises a nucleotide sequence that is complementary to a nucleotide sequence in a target nucleic acid molecule or a portion thereof and the sense strand comprises a nucleotide sequence corresponding to the target nucleic acid sequence or a portion thereof. The siNA can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region comprises a nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to a nucleotide sequence in a target nucleic acid molecule or a portion thereof and the sense region comprises a nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single-stranded polynucleotide having a nucleotide sequence complementary to nucleotide sequence in a target nucleic acid molecule or a portion thereof (for example, where such siNA molecule does not require the presence within the siNA molecule of a nucleotide sequence corresponding to the target nucleic acid sequence or a portion thereof), wherein the single-stranded polynucleotide can further comprise a terminal phosphate group, such as a 5′-phosphate (see for example, Martinez et al., 2002, Cell, 110, 563-574 and Schwarz et al., 2002, Molecular Cell, 10, 537-568), or 5′,3′-diphosphate.

siRNA directs the sequence-specific silencing of mRNA through a process known as RNA interference (RNAi). The process occurs in a wide variety of organisms, including mammals and other vertebrates. Methods for preparing and administering siRNA and their use for specifically inactivating gene function are known. siRNA includes modified and unmodified siRNA. Examples and a further description of siRNA can be found in WO2009/126933, which is hereby incorporated by reference.

A number of exemplary routes of delivery are known that can be used to administer siRNA to a subject. In addition, the siRNA can be formulated according to any exemplary method known in the art. Examples and a further description of siRNA formulation and administration can be found in WO2009/126933, which is hereby incorporated by reference.

### Linkers

The covalent linkages between the PSPF peptides and the oligonucleotide or siRNA of the modular composition and/or between targeting ligands and the oligonucleotide or siRNA may be optionally mediated by a linker. This linker may be cleavable or non-cleavable, depending on the application. In certain embodiments, a cleavable linker may be used to release the oligonucleotide after transport from the endosome to the cytoplasm. The intended nature of the conjugation or coupling interaction, or the desired biological effect, will determine the choice of linker group. Linker groups may be combined or branched to provide more complex architectures. Suitable linkers include those as described in WO2009/126933, which is hereby incorporated by reference.

In one embodiment, a suitable linker is selected from the group as shown in Table 4.

TABLE 4
Suitable linkers

| | | |
|---|---|---|
| 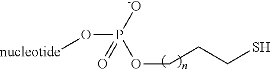 | 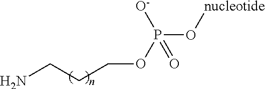 | 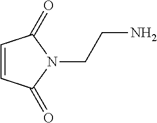 |

| | |
|---|---|
| 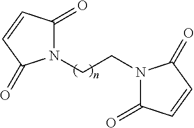 | 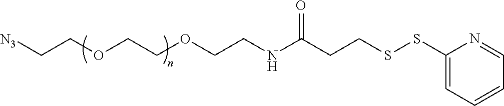 |
| 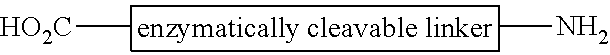 | 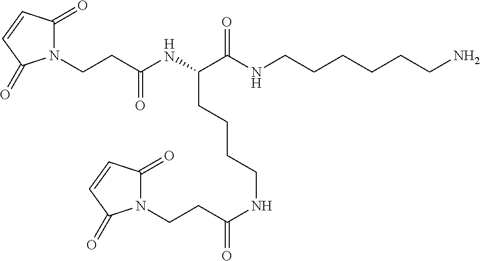 [SMILES: NCCCCCCNC(=O)[C@H](CCCCNC(=O)CCN1C(=O)C=CC1=O)NC(=O)CCN1C(=O)C=CC1=O] |

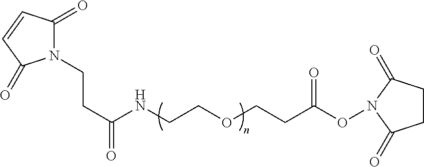

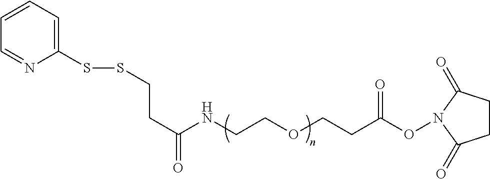

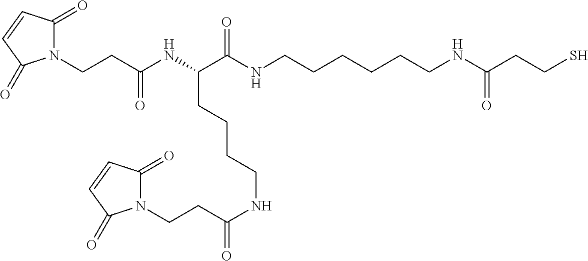 [SMILES: O=C(CCS)NCCCCCCNC(=O)[C@H](CCCCNC(=O)CCN1C(=O)C=CC1=O)NC(=O)CCN1C(=O)C=CC1=O]

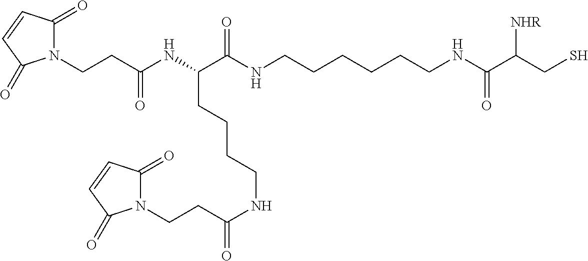

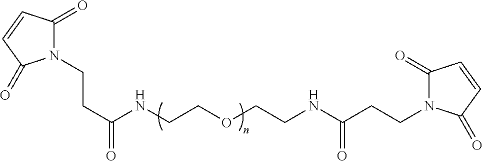

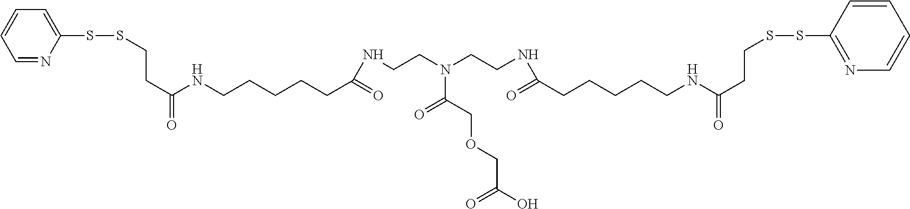 [SMILES: O=C(O)COCC(=O)N(CCNC(=O)CCCCCNC(=O)CCSSc1ccccn1)CCNC(=O)CCCCCNC(=O)CCSSc1ccccn1]

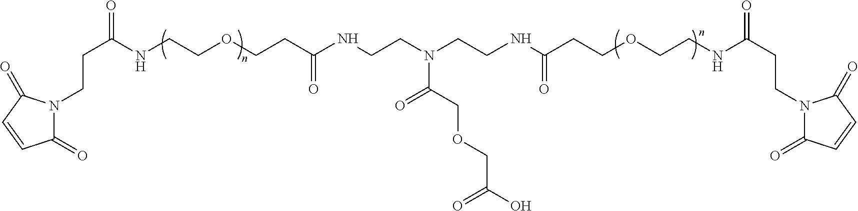

| | |
|---|---|
| 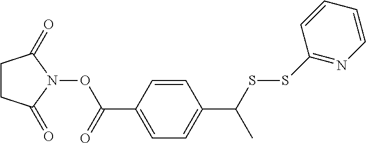 [SMILES: CC(SSc1ccccn1)c1ccc(C(=O)ON2C(=O)CCC2=O)cc1] | 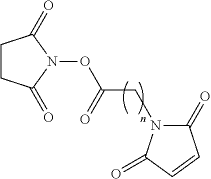 |

R = H, Boc, Cbz, Ac, PEG, lipid, targeting ligand, linker(s) and/or peptide(s).
n = 0 to 750.
“nucleotide” can be substituted with non-nucleotide moiety such as abasic or linkers as are generally known in the art.
enzymatically cleavable linker = linker cleaved by enzyme; e.g., protease or glycosidase

Commercial linkers are available from various suppliers such as Pierce or Quanta Biodesign including combinations of said linkers. In addition, commercial linkers attached via phosphate bonds or additional amino acids residues can be used independently as linkers or in combination with said linkers.

### Other Peptides

For macromolecular drugs and hydrophilic drug molecules, which cannot easily cross bilayer membranes, entrapment in endosomal/lysosomal compartments of the cell is thought to be the biggest hurdle for effective delivery to their site of action. Without wishing to be bound by theory, it is believed that the use of peptides will facilitate oligonucleotide escape from these endosomal/lysosomal compartments or oligonucleotide translocation across a cellular membrane and release into the cytosolic compartment.

In additional to the PSPF peptides disclosed herein, other peptides can also be used in the modular compositions. In one embodiment, the other peptides may be polycationic or amphiphilic or polyanionic or zwitterionic or lipophilic or neutral peptides or peptidomimetics which can show pH-dependent membrane activity and/or fusogenicity. A peptidomimetic may be a small protein-like chain designed to mimic a peptide.

In one embodiment, the other peptides are cell-permeation agents, preferably helical cell-permeation agents. These peptides are commonly referred to as Cell Penetrating Peptides. See, for example, “Handbook of Cell Penetrating Peptides” Ed. Langel, U.; 2007, CRC Press, Boca Raton, Fla. Preferably, the component is amphipathic. The helical agent is preferably an alpha-helical agent, which preferably has a lipophilic and a lipophobic phase. A cell-permeation agent can be, for example, a cell permeation peptide, cationic peptide, amphipathic peptide or hydrophobic peptide, e.g. consisting primarily of Tyr, Trp and Phe, dendrimer peptide, constrained peptide or crosslinked peptide. Examples of cell penetrating peptides include Tat, Penetratin, and MPG. It is believed that the cell penetrating peptides can be a “delivery” peptide, which can carry large polar molecules including peptides, oligonucleotides, and proteins across cell membranes. Cell permeation peptides can be linear or cyclic, and include D-amino acids, “retro-inverso” sequences, nonpeptide or pseudo-peptide linkages, peptidyl mimics. In addition the peptide and peptide mimics can be modified, e.g. glycosylated, pegylated, or methylated. Examples and a further description of peptides can be found in WO2009/126933, which is hereby incorporated by reference. Synthesis of peptides is well known in the art.

The peptides may be conjugated at either end or both ends by addition of a cysteine or other thiol containing moiety to the C- or N-terminus. In some instances, additional “spacer” amino acids can be used between the PSPF and the oligonucleotide attachment point. When not functionalized on the N-terminus, peptides may be capped by an acetyl group, or may be capped with a lipid, a PEG, or a targeting moiety. When the C-terminus of the peptides is unconjugated or unfunctionalized, it may be capped as an amide, or may be capped with a lipid, a PEG, or a targeting moiety.

### Targeting Ligands

The modular compositions of the present invention may optionally comprise a targeting ligand. In some embodiments, this targeting ligand may direct the modular composition to a particular cell. For example, the targeting ligand may specifically or non-specifically bind with a molecule on the surface of a target cell. The targeting moiety can be a molecule with a specific affinity for a target cell. Targeting moieties can include antibodies directed against a protein found on the surface of a target cell, or the ligand or a receptor-binding portion of a ligand for a molecule found on the surface of a target cell. Examples and a further description of targeting ligands can be found in WO2009/126933, which is hereby incorporated by reference.

In one embodiment, the targeting ligands are selected from the group consisting of an antibody, a ligand-binding portion of a receptor, a ligand for a receptor, an aptamer, D-galactose, N-acetyl-D-galactosamine (GalNAc), multivalent N-acetyl-D-galactosamine comprising 2-5 GalNAcs, D-mannose, cholesterol, a fatty acid, a lipoprotein, folate, thyrotropin, melanotropin, surfactant protein A, mucin, carbohydrate, multivalent lactose, multivalent galactose, N-acetyl-galactosamine, multivalent N-acetyl-galactosamine, N-acetyl-glucosamine, multivalent mannose, multivalent fructose, glycosylated polyaminoacids, transferin, bisphosphonate, polyglutamate, polyaspartate, a lipophilic moiety that enhances plasma protein binding, a steroid, bile acid, vitamin B12, biotin, an RGD peptide, an RGD peptide mimic, ibuprofen, naproxen, aspirin, folate, and analogs and derivatives thereof.

The preferred targeting ligands are selected from the group consisting of D-galactose, N-acetyl-D-galactosamine (GalNAc), GalNAc2, GalNAc3, GalNAc4, GalNAc5, cholesterol, folate, and analogs and derivatives thereof. As used herein, the terms “GalNAc2”, “GalNAc3”, “GalNAc4” and “GalNAc5” mean multivalent N-acetyl-D-galactosamines comprising 2, 3, 4 and 5 GalNAcs, respectively.

### Lipids

Lipids such as cholesterol or fatty acids, when attached to highly hydrophilic molecules such as nucleic acids can substantially enhance plasma protein binding and consequently circulation half life. In addition, lipophilic groups can increase cellular uptake. For example, lipids can bind to certain plasma proteins, such as lipoproteins, which have consequently been shown to increase uptake in specific tissues expressing the corresponding lipoprotein receptors (e.g., LDL-receptor or the scavenger receptor SR-B1). Lipophilic conjugates can also be considered as a targeted delivery approach and their intracellular trafficing could potentially be further improved by the combination with endosomolytic agents.

In one embodiment, the modular composition disclosed herein can optionally comprise one or more lipids. Exemplary lipids that enhance plasma protein binding include, but are not limited to, sterols, cholesterol, fatty acids, cholic acid, lithocholic acid, dialkylglycerides, diacylglyceride, phospholipids, sphingolipids, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, 03-(oleoyl)cholenic acid, dimethoxytrityl, phenoxazine, aspirin, naproxen, ibuprofen, vitamin E and biotin etc. Examples and a further description of lipids can be found in WO2009/126933, which is hereby incorporated by reference.

The preferred lipid is cholesterol.

### Method of Treatment

In one embodiment, a method of treating a subject at risk for or afflicted with a disease that may benefit from the administration of the modular composition of the invention. The method comprises administering the modular composition of the invention to a subject in need thereof, thereby treating the subject. The PSPF peptides and/or oligonucleotides that are administered will depend on the disease being treated. See WO2009/126933 for additional details regarding methods of treatments for specific indications.

Formulation

There are numerous methods for preparing conjugates of oligonucleotide compounds. The techniques should be familiar to those skilled in the art. A useful reference for such reactions is Bioconjugate Techniques, Hermanson, G. T., Academic Press, San Diego, Calif., 1996. Other references include WO2005/041859; WO2008/036825 and WO2009/126933.

### EXAMPLES

The invention is further illustrated by the following examples, which should not be construed as further limiting. The contents of all references, pending patent applications and published patents, cited throughout this application are hereby expressly incorporated by reference.

### Biological and Biophysical Assays

### Hemolysis Assay

Human Red Blood hemolysis assay was carried out as described below.

About 5 ml human blood from healthy individuals were transferred into a 50 ml centrifuge tube and either re-suspended in 35 ml buffer pH 5.4 (150 mM NaCl, 20 mM MES) or pH 7.5 (150 mM NaCl, 20 mM Hepes). Red Blood Cells (RBCs) were washed 3 times with the appropriate buffer and finally re-suspended in a total of 50 ml buffer (pH 5.4 or 7.5). For the assay 175 μl of buffer solution (pH 5.4 or 7.5) was added into each well of a clear-bottom 96-well plate followed by 50 μl of re-suspended RBCs (approx. 2.5×107 cells) in the appropriate buffer (for RBC transfer wide bore pipet tips were used to avoid cell damage). Test PSPF peptides (New England Peptide TM) at the appropriate concentration were diluted in 25 μl PBS and then added to the cells. All steps were done with chilled buffers and on ice. The suspension was then mixed 6-8 times by pipetting with wide bore tips, the plate was covered and incubated at 37° C. for indicated time.

After incubation the cells were centrifuged for 5 min at 500×g and 150 μl of the supernatant was transferred into a new 96-well clear-bottom plate. Absorbance at 541 nm was measured and hemolysis was normalized to RBCs which have been incubated in the presence of 1% Triton X-100 (100% hemolysis).

### Micro-RNA Mir-16

The release of micro-RNA mir-16 from RBCs was determined using stem-loop PCR as described below.

About 5 μl of supernatant was processed with TaqMan MicroRNA Cells-to-CT Kit (Applied Biosystems) according to manufacturers' protocol and quantitative PCR reaction was performed on an ABI (Applied Biosystems) 7500 Fast Real Time PCR System using standard cycling conditions 37. The derived Ct values for mir-16 (Applied Biosystems cat. no.: 4373121) in each experiment were transformed into copy numbers using a linear equation derived from a standard curve which was run in parallel.

### ATP

To quantitatively determine the amount of Adenosine TriPhosphate (ATP) in the supernatant, the ATPLite assay kit (Perkin Elmer; Waltham, Mass.) was used according to the manufacturers' instructions using 100 μl supernatant per reaction point.

### Tryptophan Fluorescence Excitation Wavelength

The fluorescence spectra were collected on a Fluorolog spectrofluorometer. The tryptophan fluorescence of each peptide was measured at both pH 5.5 and pH 7.4 (30 m M Phos and 150 m M NaCl), with and without lipid titration. The lipid stock was prepared with 90% POPC and 10% POPG, and the final concentration of lipid after titration is 200 μM. The peptide concentration in each measurement was 2 μM.

### Circular Dichrosim (CD) Measurement and Thermal Denaturation

CD spectra were collected with a Jasco J-810 spectropolarimeter using a 1-nm step at 4° C., at both pH 5.5 and pH 7.4 (30 m M Phos and 150 m M NaCl). The PSPF-EKG peptide concentration was 2 μM. The CD spectrum was obtained by averaging over three scans.

The helical CD signal at 222 nm for 2 μM, 4 μM and 20 μM was monitored as temperature increased from 4° C. to 96° C. at both pH 5.5 and pH 7.4 (30 m M Phos and 150 m M NaCl), in a 2° C. steps. The parameters from the Gibbs-Helmholtz Equation were fit to the data.

### Size Exclusion Chromatography

Size exclusion chromatography (SEC) of 100 μM PSPF-EKG and 100 μM PSPF-DKG were measured by AKTA FPLC machine (GE) using a Superdex 75 column (GE) eluted at pH 7.4 (50 mM Tris, 150 mM NaCl) and pH 5.5 (50 mM MES, 150 mM NaCl) respectively, at 25° C. Four standards were used: blue dextran (2,000,000 g/mol), carbonic anhydrase (29,000 g/mol), cytochrome C (12,400 g/mol) and aprotinin (6,500 g/mol). In order to test the effect of salt concentration upon peptide elution, the elutions of PSPF-EKG were also measured at pH 7.4 (50 mM Tris, 2M NaCl) and pH 5.5 (50 mM MES, 2 NaCl), respectively.

### Sedimentation Equilibrium of Analytical Ultracentrifugation (AUC)

Sedimentation Equilibrium of Analytical Ultracentrifugation (AUC) of 100 μM PSPF-EKG was measured at 25° C. using a Beckman XL-I analytical ultracentrifuge at 35, 40, 45, and 50 kRPM, at both pH 7.4 (50 mM Tris, 150 mM NaCl) and pH 5.5 (50 mM MES, 150 mM NaCl). The data was globally fit to a nonlinear least squares curves by IGOR Pro (Wave-metrics) as previously demonstrated.

The AUC measurement of PSPF-EKG has also been measured in N-tetradecyl-N,N dimethyl-3-ammonio-1-propanesulfonate (C-14 betaine) micelles. 17% D₂O in buffer was used to precisely match the density of 8 mM C-14 betaine micelle at pH 7.4 (50 mM Phos, 150 mM NaCl) and 22% D₂O was used for pH 5.5 (50 mM Phos, 150 mM NaCl). Three groups of samples were prepared as peptide:DPC molar ratios of 1:50, 1:100, and 1:200 at both pHs. The data with three peptide/detergent ratios and four rotor speeds (35, 40, 45, and 50 kRPM) was globally fit to a nonlinear least squares curves by IGOR Pro (Wave-metrics) as previously demonstrated.

### Attenuated Total Reflection IR Spectroscopy (ATR-IR)

ATR-IR of PSPF-EKG was measured by a Nicolet Magna IR 4700 spectrometer using 1 cm⁻¹ resolution. About 5.0⁻⁷ mole PSPF-EKG in trifluoroethanol (TFE) was mixed with 20 fold mole of 1-palmitoyl-2-oleoyl-snglycero-3-phosphocholine (POPC) and dried into a thin film on the surface of ATR Ge crystal evenly by N₂ gas. The film was rehydrated by D₂O-saturated air overnight in closed environment of D₂O bath. During data acquisition, the polarized mirror was adjusted to 0° and 90°, creating incident light oriented parallel and perpendicular to the lipid normal respectively. The infrared spectrum of each condition was averaged over 256 scans. The dichroic ratio of 1656 cm⁻¹ amide I bond absorption is computed for parallel (0°) versus perpendicular (90°) polarized incident light relative to the membrane normal and has been used to calculate the peptide orientation as previously shown.

### Example 1

### Cellular Release Assays

Red blood cell (RBC) lysis assays was used to screen the functional efficacy of the PSPF peptides upon delivery (Table 5). The release of ATP, miRNA and hemoglobin has been studied at both pH 7.5 and 5.4. The peptide was designed to selectively deliver the nucleotides or ribonucleic acid, with sizes similar to ATP and miRNA, across the membrane only at pH 5.5.

A desirable peptide should also negate membrane disruption, as assessed by leakage of proteins such as hemoglobin at both pH values. Therefore the peptides were first screened for hemolytic activity at both pH 7.5 and pH 5.4. None of the twelve peptides SEQ ID No. 1-12 had hemolytic activity at either pHs. When screening for ATP and miRNA release at 5 μM, PSPF-DQA, PSPF-DKG, and PSPF-EKG showed relatively high release percentage for ATP (more than 20%) and miRNA (more than 10%) at pH 5.4, and also low release percentage at pH 7.5 for both ATP and miRNA (less than 10%). Among the top three peptides screened out of RBC assays, PSPF-EKG was further characterized to reveal the mechanism of action.

TABLE 5
RBC Lysis Assay of PSPF Peptides

| | RBC Lysis Assay (% calculated compared to triton-x-100) | | | | | |
|---|---|---|---|---|---|---|
| | Hemoglobin | | % ATP | | % miRNA | |
| | Release | | at 5 μM | | at 5 μM | |
| Peptide | pH 7.5 | pH 5.4 | pH 7.5 | pH 5.4 | pH 7.5 | pH 5.4 |
| PSPF-DQA | none | none | 3.81 | 17.91 | 0.81 | 18.61 |
| PSPF-DQG | none | none | 3.21 | 8.61 | 0.06 | 0.16 |
| PSPF-DKA | none | none | 4.64 | 5.79 | 4.13 | 0.79 |
| PSPF-DKG | none | none | 7.54 | 24.1 | 5.54 | 7.47 |
| PSPF-EQA | none | none | 3.69 | 3.61 | 0.46 | 0.02 |
| PSPF-EQG | none | none | 3.36 | 9.52 | 0.15 | 2.64 |
| PSPF-EKA | none | none | 2.02 | 3.66 | 0.51 | 0.21 |
| PSPF-EKG | none | none | 3.38 | 27.3 | 0.14 | 12.54 |
| PSPF-HQA | none | none | 6.17 | 11.72 | 0.02 | 1.43 |
| PSPF-HQG | none | none | 5.69 | 10.55 | 0.2 | 0.64 |
| PSPF-HKA | none | none | 0.93 | 5.7 | 0.43 | 0.02 |
| PSPF-HKG | none | none | 32.1 | 39.22 | 72.28 | 0.44 |

### Example 2

### Peptide Engagement with the Lipid Bilayer by Tryptophan Fluorescence

To detect the engagement of PSPF peptides with lipid vesicles, tryptophan (Trp) fluorescence was measured for PSPF-DQA, DKG and EKG. The extent of environmental change around the N-terminal Trp was determined by the observed shift and changes in intensity of the fluorescence signal. Blue shifts correspond to a more hydrophobic environment, such as that which would occur to the Trp upon membrane interaction or insertion. The majority of the PSPF-peptides studied showed minimal blue shifting at pH 7.4 and larger shifts at pH 5.5 (Table 6). PSPF-DQA showed small detectable shift at pH 5.5 (−1 nm), whereas PSPF-DKG and EKG showed blue shifts of approximately 3 nm (350 to 347 nm) each at pH 5.5. PSPF-HKG also showed a significant shift from 351 to 341 nm at pH 5.5.

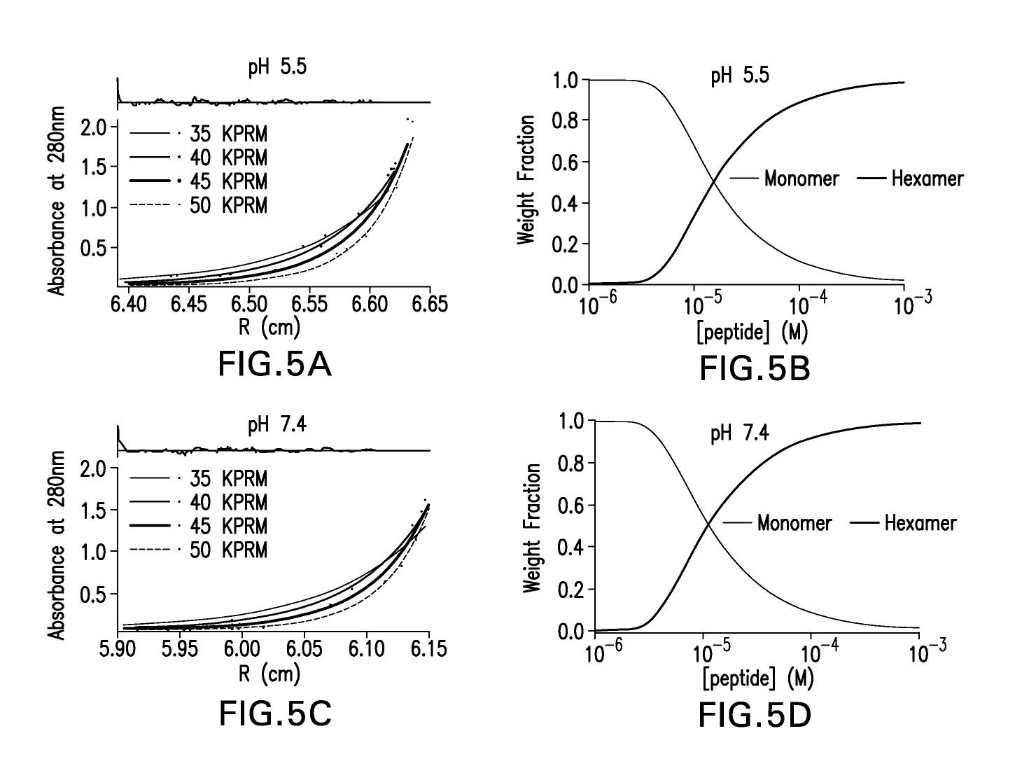

Despite different experimental conditions, Trp fluorescence shifts among all peptides correlated strongly with ATP release at pH 5.5, with R2 of 0.74 if linear regression is applied (FIG. 5). At pH 5.5, a larger shift in Trp fluorescence (likely due to insertion into the membrane of Trp) corresponded to greater release of ATP (likely from membrane insertion and pore formation). This suggests that the peptides were acting in a similar manner in both experimental assays and consistent with pH-sensitive insertion and pore formation.

TABLE 6
Trp fluorescence of PSPF- series peptides
with various amounts of lipid vesicles

| | pH 7.4 | | | | pH 5.5 | | | |
|---|---|---|---|---|---|---|---|---|
| | λₘₐₓ (nm) | | | % | λₘₐₓ (nm) | | | % |
| Pep- | 0 | 200 | Δ | Inten- | 0 | 200 | Δ | Inten- |
| tide | μM | μM | λₘₐₓ | sity In- | μM | μM | λₘₐₓ | sity In- |
| PSPF- | Lipid | Lipid | (nm)^{#} | crease* | Lipid | Lipid | (nm) | crease |
| DQA | 352 | 351 | −1 | 32 | 348 | 347 | −1 | 38 |
| DQG | 354 | 353 | −1 | 18 | 350 | 358 | −2 | 33 |
| DKA | 354 | 353 | −1 | 18 | 349 | 358 | −1 | 38 |
| DKG | 355 | 351 | −4 | 36 | 350 | 347 | −3 | 38 |
| EQA | 352 | 352 | 0 | 6 | 349 | 348 | −1 | 21 |
| EQG | 355 | 354 | −1 | 15 | 349 | 346 | −3 | 42 |
| EKA | N/A | N/A | N/A | N/A | N/A | N/A | N/A | N/A |
| EKG | 354 | 352 | −2 | 28 | 350 | 347 | −3 | 52 |
| HQA | N/A | N/A | N/A | N/A | N/A | N/A | N/A | N/A |
| HQG | N/A | N/A | N/A | N/A | N/A | N/A | N/A | N/A |
| HKA | N/A | N/A | N/A | N/A | 349 | 347 | −2 | 34 |
| HKG | N/A | N/A | N/A | N/A | 351 | 341 | −10 | 72 |

^{#}Δ λₘₐₓ = λₘₐₓ at 200 μM Lipid − λₘₐₓ at 0 μM Lipid
*% Intensity Increase = (Intensity at 200 μM Lipid − Intensity at 0 μM Lipid)/Intensity at 0 μM Lipid

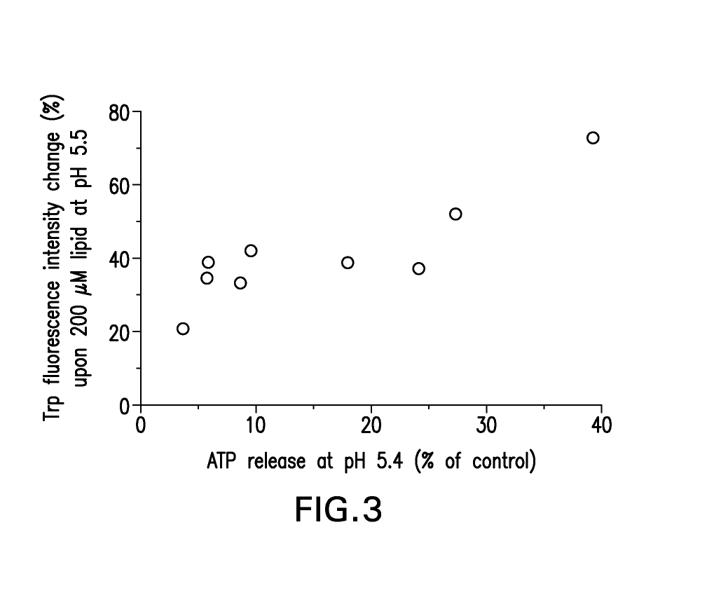

FIG. 3 shows the correlation between ATP-release by PSPF peptides and the degree of lipid engagement as assessed by the fractional change of Trp-fluorescence signal upon addition of 200 μM lipid vesicles.

### Example 3

### The Association Properties of PSPF Peptides in an Aqueous System

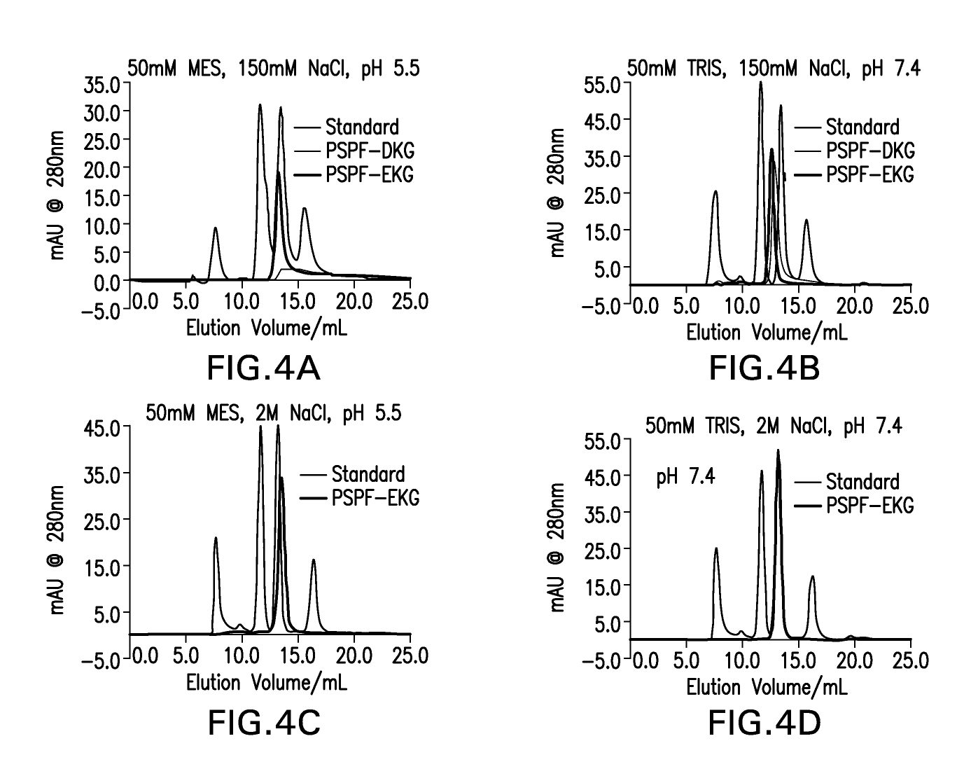

Size exclusion chromatography—The association state of the PSPF peptide PSPF-EKG was initially investigated by size exclusion chromatography (SEC) using a Superdex 75 column (GE Healthcare) eluted at pH 7.4 (150 mM NaCl, 50 mM Tris) and pH 5.5 (150 mM NaCl, 50 mM MES) respectively. In addition, PSPF-DKG was also investigated to determine the effect of substituting Asp for Glu on the stability of the water-soluble bundle at each pH. To determine the approximate oligomerization states, four standards were used, shown by blue eluting peaks in FIG. 4: blue dextran (2,000,000 g/mol), carbonic anhydrase (29,000 g/mol), cytochrome C (12,400 g/mol) and aprotinin (6,500 g/mol).

PSPF-EKG eluted with an apparent molecular weight 6.5-fold higher than the calculated molecular weight at pH 7.4 and 5.2-fold at pH 5.5 (FIG. 4, Table 7), both as a single species. Noticeably PSPF-EKG presented a peak with significantly lower intensity and a broad trailing feature when eluting at pH 5.5, indicating that the decreased pH has increased the propensity to interact with column, which may act as a mimic of the membrane phase (FIG. 4A). Dissociation during elution might also contributed to the peak shape, indicative of a lower stability of the water-soluble helical bundle. Similarly, PSPF-DKG eluted with an apparent molecular weight 6.0-fold higher than the calculated molecular weight at pH 7.4 as a single species and nearly failed to elute at pH 5.5 (FIG. 4A), indicating the lower pH drove the peptide to interact with the column. Furthermore, when the salt concentration was increased to 2M, the shoulder of elution peak for PSPF-EKG still existed at pH 5.5 (FIGS. 4C, D). Also, Asp at the putative “a” position made the PSPF-DKG more sensitive to the pH decrease than Glu in PSPF-EKG, in terms of driving the peptide's preference away from the aqueous phase (FIG. 4A).

FIG. 4 shows the size exclusion chromatography of PSPF-EKG and PSPF-DKG at each pH. Both PSPF-EKG and PSPF-DKG eluted as a single species corresponding to the oligomerization of hexamer at pH 7.4 (B). PSPF-EKG eluted as a single-species peak with a significant shoulder at pH 5.4 and the major peak corresponded to a formation of hexamer. PSPF-DKG almost failed to elute at PH 5.5 (A). The salt concentration was increased to 2M and the shoulder of elution peak still existed at pH 5.5 (C, D).

TABLE 7
Apparent molecular weight and calculated oligomerization
state based on size exclusion chromatography for PSPF-EKG
and PSPF-DKG at both PHs

| | PSPF-EKG | | PSPF-DKG | |
|---|---|---|---|---|
| | pH 7.4 | pH 5.5 | pH 7.4 | pH 5.5 |
| Apparent MW | 19,000 | 15,000^{#} | 17,000 | N/A |
| Oligomerization State* | 6.6 | 5.2 | 6.0 | N/A |

*Oligomerization State = Apparent MW/Monomer MW;
^{#}Major peak

### Example 4

### Sedimentation Equilibrium of Analytical Ultracentrifugation

Analytical ultracentrifugation (AUC) sedimentation equilibrium was applied to further investigate the association state and affinity of the water-soluble bundles of both PSPF-EKG and PSPF-DKG. The peptides were studied at 100 μM peptide concentration and pH 7.4 (150 mM NaCl, 50 mM Tris) or pH 5.5 (150 mM NaCl, 50 mM MES). The parameters were globally fit to data collected over multiple rotor speeds (35, 40, 45, 50 KRPM). Fitting the curve to a single MW species suggested apparent molecular weights for PSPF-EKG of 18,000±30 at pH 7.4 (FIG. 5A) and 16,000±30 at pH 5.5 (FIG. 5B). This agrees well with the data from size exclusion chromatography and points to a hexameric association state at both pHs for PSPF-EKG. The data can be further fit to a monomer-hexamer equilibrium, resulting in an association energy ΔG of −6.3 kcal/mol monomer at pH 7.4 and −5.6 kcal/mol monomer at pH 5.5 (Table 8). Also, as shown in the plot of species weight fraction, the concentration of peptide required to associate at pH 7.4 was lower than at pH 5.5 (FIG. 5B, D). Together it suggests that decreased pH destabilized the helix bundle of PSPF-EKG.

FIG. 5 shows the AUC sedimentation equilibrium of PSPF-EKG at pH 5.5 (A) and 7.4 (C). Single species fitting of PSPF-EKG suggests a hexameric association state at both pH 7.4 (A) and pH 5.5 (C). For each peptide and pH condition, the top plot shows the single species fitting with residuals above while the below plot shows the species weight fraction. Then the data has been fit with a monomer-hexamer equilibrium model at both pHs. The dissociation state and dissociation energy is shown in Table 8. The weight fraction distributions have also been plot for pH 5.5 (B) and pH 7.4 (D).

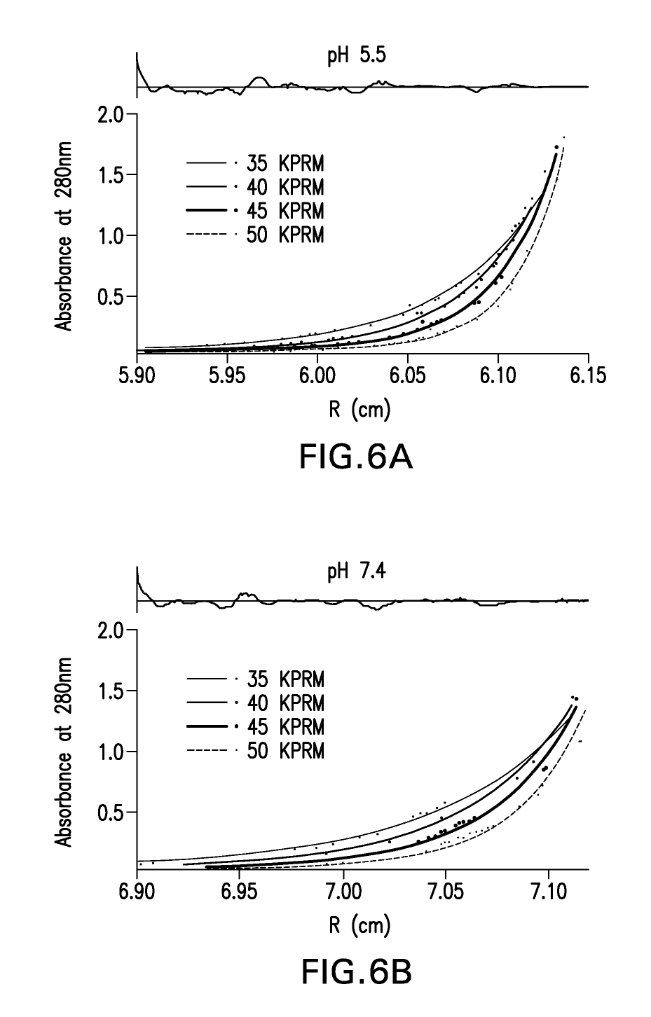

For PSPF-DKG, a global fit resulted in a single-species apparent molecular weight of 17,000±30 at pH 7.4 (FIG. 6), which was 6.0-fold higher than the calculated molecular weight and again agrees well with size exclusion chromatography. The equilibrium of PSPF-DKG has also been fit into the equilibrium of monomer-hexamer with association energy ΔG of −6.5 kcal/mol monomer (Table 8). The single-species apparent molecular weight for PSPF-DKG at pH 5.5 was 24,000±60 (FIG. 6B). This could represent a heterogeneous set of association states, taken together with the broad elution peak observed in the size exclusion chromatography.

FIG. 6 shows AUC sedimentation equilibrium of PSPF-DKG at pH 5.5 and 7.4. Single species fitting of PSPF-DKG suggests it associated as a hexamer at pH 7.4 and reached an apparent molecular weight of approximately 24,000 at pH 5.5. For each peptide and pH condition, the top plot shows the single species fitting with residuals above while the below plot shows the species weight fraction.

TABLE 8
Analytical ultracentrifugation (AUC) sedimentation equilibrium
for PSPF-EKG and PSPF-DKG at pH 5.5 and 7.4

| | PSPF-EKG | | PSPF-DKG | |
|---|---|---|---|---|
| | pH 7.4 | pH 5.5 | pH 7.4 | pH 5.5 |
| Apparent MW | 18,000 ± 30 | 16,000 ± 30 | 17,000 ± 30 | 24,000 ± 60 |
| Oligomerization State* | 6.2 | 5.5 | 6.0 | N/A |
| −log(Kdissociation) | 28.0 ± 0.4 | 24.8 ± 0.1 | 28.7 ± 0.4 | N/A |
| Association ΔG# | −6.3 | −5.6 | −6.5 | N/A |
| (kCal/mol monomer) | | | | |

*Oligomerization State = Apparent MW/Monomer MW
#Association ΔG = 2.303 * RT * log(Kdissociation)/6

### Example 5

### Circular Dichroism and Thermal Denaturing

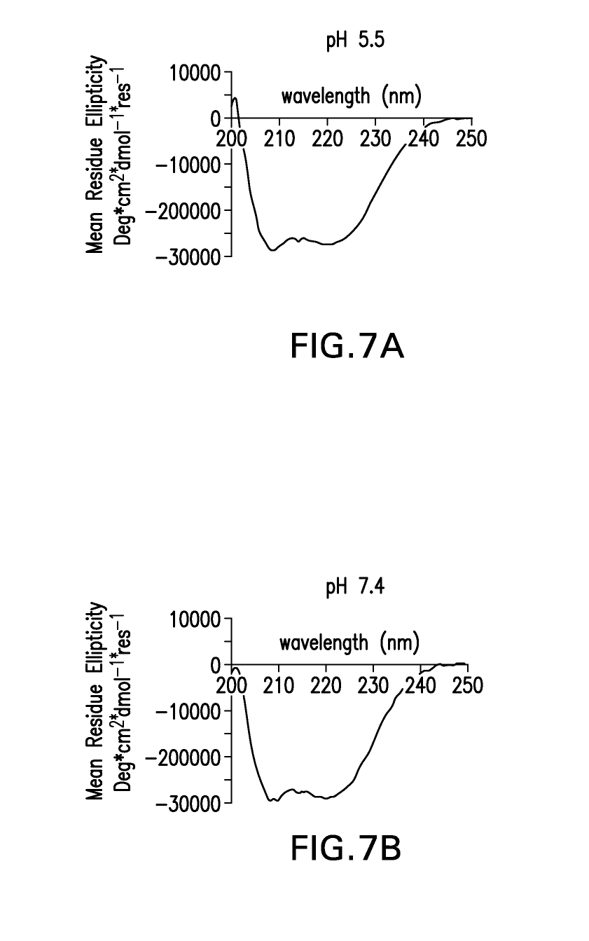

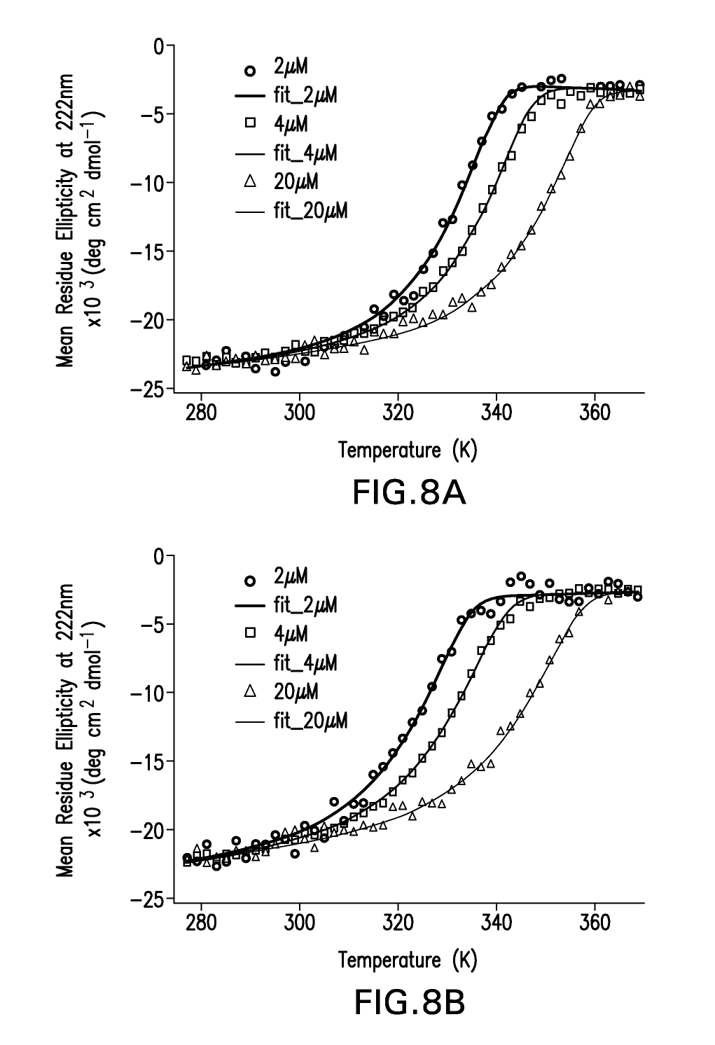

Circular dichroism (CD) suggests that PSPF-EKG adopted an alpha-helical secondary structure at both pHs (FIG. 7). Furthermore, thermal denaturation by circular dichroism (CD) was used to study the thermal stability of the PSPF-EKG hexamer at multiple concentrations (2 μM, 4 μM and 20 μM), and at both pH 7.4 (FIG. 8A) and pH 5.5 (FIG. 8B). For each pH, to the curves were analyzed according to the Gibbs-Helmholtz Equation, using global least squares fitting of ΔHm, Tm and baselines. Tm was chosen as a global parameter defined with a reference concentration of 4 μM. ΔCp was also included, but over the range of experimental data examed, this parameter was not well defined.

ΔG=ΔHm(1−T/Tm)−ΔCp[Tm−T+T[ln(T/Tm)]] Gibbs-Helmholtz Equation:

Here ΔG refers to the unfolding energy upon thermal denaturation, T refers to temperature, Tm refers to the melting temperature at which AG equals to zero. ΔHm refers to the enthalpy at Tm, and ΔCp refers to the change in the heat capacity over the temperature range.

The enthalpy at pH 7.4 is 22.0 kcal/mol monomer and is approximately 12% higher than at pH 5.5 (19.6 kcal/mol monomer) (Table 9). The values of enthalpy at both pHs are typical for designed water-soluble helix bundles. The melting temperature Tm is 339.0 K at pH 7.4 and is 5.6 K higher than at pH 5.5 (333.4K). The concentration of PSPF-EKG required to have 50% of the total amount of peptide remain folded at 300K was calculated to be 0.31 μM at pH 5.5, which was approximately double the concentration of peptide required for 50% folding at pH 7.4 (0.14 μM). These data suggests that decrease in pH destabilized the folding of PSPF-EKG.

TABLE 9
Fitting results for CD thermal denaturation of
PSPF-EKG at both pH 7.4 and pH 5.5.

| | ΔH (kcal/mol | | [PSPF-EKG] at |
|---|---|---|---|
| pH | monomer) | Tm (K) | 50% fold and 300 K |
| 7.4 | 22.0 ± 0.1 | 339.0 ± 0.1 | 0.14 μM |
| 5.5 | 19.6 ± 0.1 | 333.4 ± 0.1 | 0.31 μM |

### Example 6

### The Structural Properties of PSPF-Peptides in a Membrane Micelle System

Sedimentation equilibrium of analytical ultracentrifugation—AUC sedimentation equilibrium of PSPF-EKG in detergent micelles pointed to a weak oligomerization at both pHs. PSPF-EKG was dissolved in N-tetradecyl-N,N dimethyl-3-ammonio-1-propanesulfonate (C-14 betaine) micelles. The density of the solution was adjusted by D₂O to precisely match that of the C-14 betaine detergent at both pH 7.4 and pH 5.5 (50 mM sodium phosphate and 150 mM NaCl), so that only the peptide component contributed to the sedimentation equilibrium.

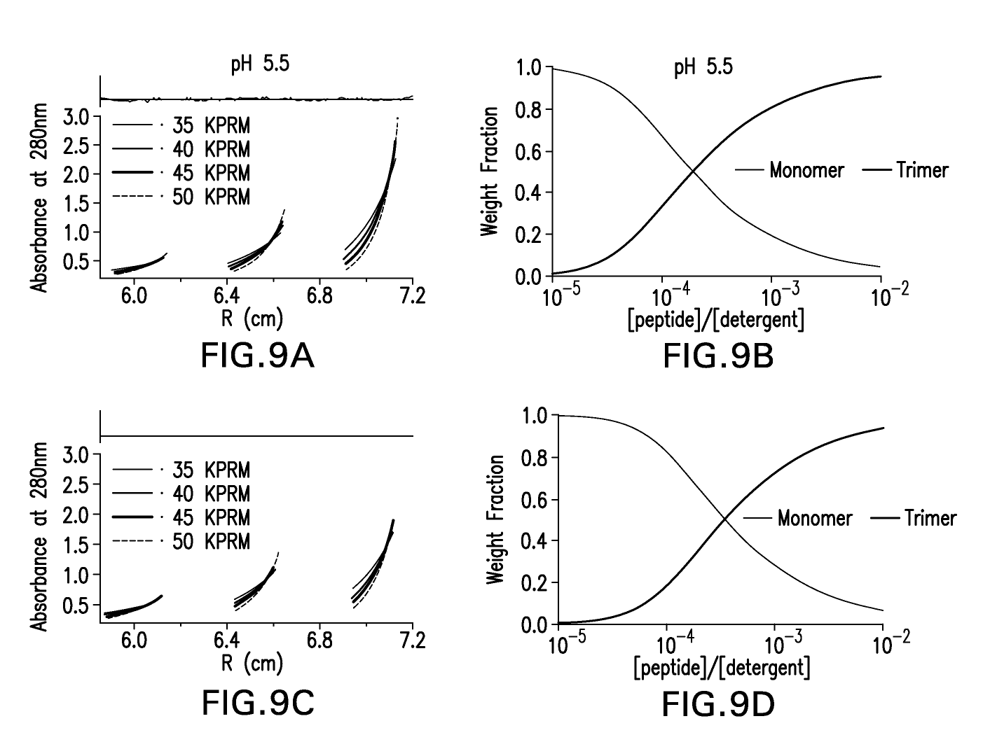

Three samples prepared at different peptide-to-detergent ratios (1:50, 1:100, 1:200) were each centrifuged at four rotor speeds (35, 40, 45, 50 KMRP) at each pH. The data could be fit into a monomer-trimer, monomer-tetramer, and monomer-higher oligomer equilibrium, suggesting that PSPF-EKG weakly associated in detergent micelle. FIG. 9 showed an example in which a monomer-trimer equilibrium was fit to the data at pH 7.4 (FIG. 9A) and pH 5.4 (FIG. 9C), and the weight fraction distribution was shown in FIGS. 9B and 9D.

### Example 7

### The Orientation of PSPF-EKG in a Lipid Bilayer

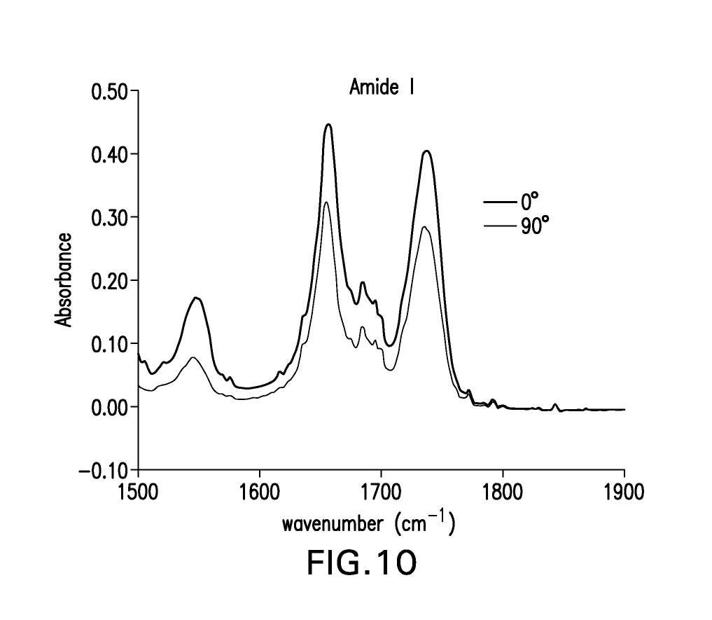

Attenuated total reflection IR spectroscopy—The secondary structure and orientation of PSPF-EKG in deuterium oxide (D₂O) hydrated bilayers were evaluated using attenuated total reflection IR spectroscopy (ATR-IR). The IR spectra in the amide I region of the PSPF-EKG showed a single peak at 1656 cm⁻¹, indicative of a dehydrated helical conformation in bilayers (FIG. 10). The dichroic ratio for parallel versus perpendicularly polarized light was 1.5, corresponding to an order parameter of −0.42. This order parameter would correspond to an orientation of approximately 75° relative to the membrane normal, assuming the bilayers were well ordered and the entire peptide fully helical. The result suggests that the majority of peptide lies parallel to the lipid surface, and rules out the possibility of the peptide being oriented predominantly perpendicular to the bilayer surface. The fact that the computed angle is less than 90° is also consistent with a small amount of peptide adopting a vertically inserted conformation, in equilibrium with the predominant form, although other models could also lead to the observed 75° angle.

FIG. 10 shows the ATR-IR of PSPF-EKG in phospholipids (POPC) bilayers. The peak at 1656 cm⁻¹ is indicative of alpha helical secondary structure. The orientation is demonstrated by the ratio of peak area of the 1656 cm⁻¹ amide I bond for parallel (0°) versus perpendicular (90°) polarized incident light (relative to the membrane normal).

In one embodiment, the RBC lysis assay on PSPF-EKG showed highest target molecule delivery efficiency at selective pH (5.4). Lack of hemolytic activity ruled out the possibility of undesirable membrane description by PSPF-EKG at both pHs. Also, the nice correlation between ATP release at pH 5.5 and Trp-fluorescence at pH 5.4 upon lipid titration (FIG. 3), indicates that membrane insertion presumably played a key role in ATP release.

RBC Lysis data also provided a direct comparison among peptides of SEQ. ID No. 1-12. Firstly, there are three options of pH-trigger residues in this peptide series. Asp and Glu residues both presented expected pH-switchable ATP and miRNA release in peptides PSPF-DQA and PSPF-DKG, indicating the carboxyl side chain groups responded efficiently to environmental pH change, though their intrinsic pKa of the unperturbed side chain is around 4. The third trigger candidate, His, failed to show significant pH preferences in terms of ATP or miRNA release. However PSPF-HKG induced high ATP release percentage at both pHs. Presumably His will induce pore formation in a pH-independent manner. Nevertheless, all the His variants ran into solubility issues in the further biophysical characterization and thus were not considered as preferred candidates for further pharmaceutical development.

Lys and Gln were in “f” positions in order to provide helix propensities in aqueous system and solvent exposure surface in membrane system. The RBC lysis results did not discriminate between these two residues when comparing the performance of the aspartate and glutamate peptide variants (PSPF-EKG versus PSPF-EQG, PSPF-EKA versus PSPF-EQA).

The choice of Ala or Gly was studied for residues packed in the helix interface. This part of the design was in light of previously discovered fact that small residues were preferred in TM helix interaction interface to stabilize the final folded state (TM helix bundle). In the case of PSPF-EKG versus PSPF-EKA, Gly resulted in a much higher pH-switchable ATP and miRNA release. The results agreed with the previous conclusion that Gly in TM helical interface drove stronger TM helix association that Ala, presumably because Gly stabilized the helix interaction via weak Cá—H interaction.

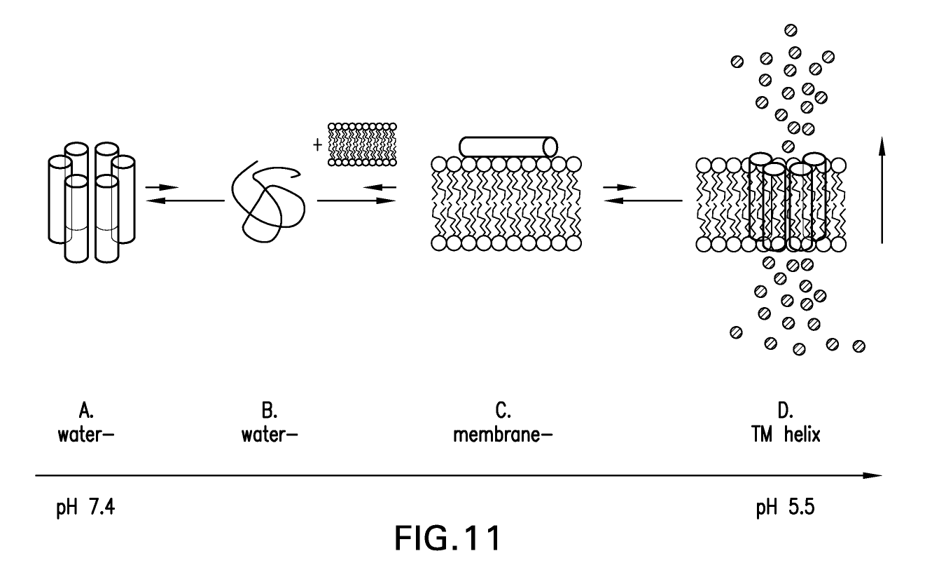

A variety of biophysical assays have been applied to obtain a comprehensive mechanism of PSPF-EKG's pH switchable pore formation. The structural conformation and folding stability of PSPF-EKG in aqueous solution was studied and CD, AUC and SEC suggest that PSPF-EKG formed a stable helix bundle at both pHs (FIG. 11A), which is expected due to the designed canonical Leu-zipper coiled-coil motif. AUC and thermal denaturing have been further used to study the folding stability difference between pH 7.4 and pH 5.5. The free energy of helix bundle has increased by 0.7 kcal/mol monomer upon pH decrease. Both ÄCp and Tm decreased at pH 5.5 versus pH 7.4, suggesting that PSPF-EKG was better packed at higher pH. Also, in SEC PSPF-EKG presented a significant shoulder upon elution at pH 5.5 versus a sharp peak at pH 7.4. The shoulder did not disappear even as the salt concentration in buffer increased from 150 mM to 2 M. The data suggests pH decrease destabilized the stability of PSPF-EKG in aqueous system (FIG. 11A, B), thus validating the first consideration of the original design.

PSPF-EKG in micelles and bilayers was characterized. Equilibrium sedimentation AUC suggests PSPF-EKG adopted a monomer-oligomer equilibrium in C14-betaine micelles at both PHs (FIG. 11C, D). A unique oligomerization state could not be determined by AUC due to weak association. Furthermore, the orientation of PSPF-EKG has been studied by ATR-FTIR in POPC lipid bilayers. The average dichroic angle is about 75 degrees with respect to the lipid normal, revealing that the majority of peptides were in a membrane-surface-absorbed state and adopted a vertical conformation with respect to the lipid normal. This state presumably corresponds to the monomer state identified by AUC (FIG. 11C). Also, some of the peptides adopted a TM orientation, which might reflect a weakly associated oligomeric form (FIG. 11D). This dynamic equilibrium between vertical monomer in membrane-surface-absorbed state and TM oligomer state, presumably induced membrane pore formation and played a crucial role in ATP and miRNA release (FIG. 11D).

### Example 8

### Co-Transfection Assay of Peptide and siRNA

The protocol and siRNA reagent (target SSB gene) described in the following publication was followed: Bartz R, Fan H, Zhang J, Innocent N, Cherrin C, Beck S C, Pei Y, Momose A, Jadhav V, Tellers D M, Meng F, Crocker L S, Sepp-Lorenzino L, Barnett S F. Effective siRNA delivery and target mRNA degradation using an amphipathic peptide to facilitate pH-dependent endosomal escape. Biochem J. 2011; 435:475-87. The results are shown in Table 10.

TABLE 10
siRNA Co-Transfection Assay

| Peptide SEQ. ID | % RNA KD | % viable |
|---|---|---|
| 1 | 1.0 | 100 |
| 2 | 6.0 | 98 |
| 3 | 0.0 | 101 |
| 4 | 0.0 | 104 |
| 5 | −2.0 | 103 |
| 6 | −1.0 | 105 |
| 7 | 5.0 | 105 |
| 8 | 5.0 | 102 |
| 9 | 13.0 | 105 |
| 10 | 11.0 | 104 |
| 11 | 78.0 | 97 |
| 12 | 74.0 | 102 |

### Example 9

### Preparation of siRNA-Peptide Conjugates

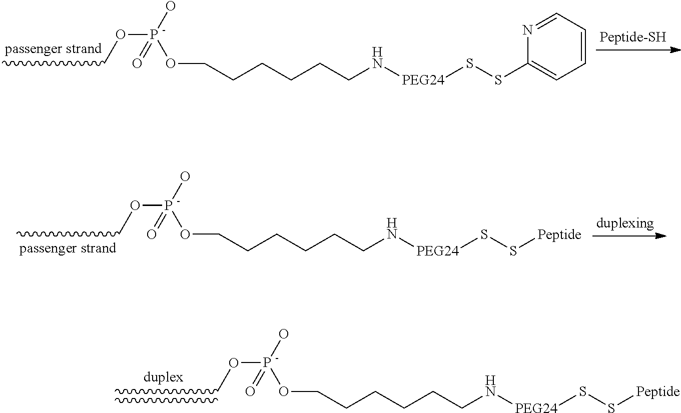

The individual peptides and oligonucleotides were prepared using standard techniques generally known in the art. The PEG24 disulfide derivative of OS1 (2 mg, 0.261 umol) was dissolved in 1 mL solution 4:1 TFE:water/50 mM CsCl/20 mM TEAA. Peptide WSDLAQALSDLAQALSDLAQALSDLAQAGGC (1.62 mg, 0.522 umol) was dissolved in 1 mL 4:1 TFE:water/50 mM CsCl/20 mM TEAA and was added to the RNA solution. The reaction was aged for 26 hours, after which RP-HPLC indicated partial conversion to product. Reaction was purified via SAX chromatography (5-50% 2:1 TFE:water with 1M CsCl, 20 mM TEA, Dionix propac column) Fractions containing product were dialyzed and lyophilized to give desired product (0.35 mg, 12.62%, uv quantified). The product (0.351 mg, 0.033 umol) was duplexed to OS2 (0.222 mg, 0.033 umol) in 28 uL water. Solution was heated to 90° C. for one minute, then cooled to RT and lyophilized to give peptide oligonucleotide duplex conjugate. A similar protocol was followed for the other peptides outlined above.

Oligo Sequence 1 (OS1)=[omeA][omeC]AA[omeC][omeU]GA[omeC][omeU][omeU][omeU]AA[omeU]G[omeU]AA[6amiL]
Oligo Sequence 2 (OS2)=[p][fluU][fluU]A[fluC]A[fluU][fluU]AAAG[fluU][fluC][fluU]G[fluU][fluU]G[fluUs][rUs]U

These examples are used as illustration only. One skilled in the art would readily appreciate that the present invention is well adapted to carry out the objects and obtain the ends and advantages mentioned, as well as those inherent therein. The methods and compositions described herein, as presently representative of preferred embodiments, are exemplary and are not intended as limitations on the scope of the invention. Changes therein and other uses will occur to those skilled in the art, which are encompassed within the spirit of the invention, are defined by the scope of the claims.

What is claimed is:

1. A pH-switchable pore formation (PSPF) peptide comprising one or more amino acids in peptide sequence whose charge state and hydrophobicity are pH-dependent, wherein the peptide can bind to a biological membrane upon contact and form pores on the membrane at pH of less than about 7, and wherein the peptide forms substantially no pores on the biological membrane at pH of greater than about 7.

2. The PSPF peptide of claim 1, wherein the peptide can bind to a biological membrane upon contact and form pores on the membrane at pH of less than 6.5, and wherein the peptide forms substantially no pores on the biological membrane at pH of greater than 7.0.

3. The PSPF peptide of claim 1, wherein the peptide can bind to a biological membrane upon contact and form pores on the membrane at pH of about 5.5, and wherein the peptide forms substantially no pores on the biological membrane at pH of about 7.4.

4. The PSPF peptide of claim 1, wherein the peptide is water soluble at pH of greater than about 7.

5. The PSPF peptide of claim 1, wherein the amino acid is selected from the group consisting of Asp, Glu and His.

6. The PSPF peptide of claim 1, wherein the pores formed on the membrane serve as channels for transport of appropriately-sized target; and wherein uptake of the peptide by endocytosis allows endosomal escape of material present in the extracellular environment into the cell.

7. The PSPF peptide of claim 1, wherein the peptide is selected from peptides of SEQ. ID No. 1-24.

8. The PSPF peptide of claim 7, wherein the peptide is selected from peptides of SEQ. ID No. 4, 8 and 12.

9. The PSPF peptide of claim 1 having a heptad repeat structure as shown in FIG. 2, wherein positions “b” in water is an amino acid selected from the group consisting of Ser and Thr.

10. The PSPF peptide of claim 1 having a heptad repeat structure as shown in FIG. 2, wherein position “c” in water is an amino acid selected from the group consisting of Asp, Glu and His.

11. The PSPF peptide of claim 10, wherein position “c” in water is Glu.

12. The PSPF peptide of claim 1 having a heptad repeat structure as shown in FIG. 2, wherein each of positions “e” and “g” is an amino acid independently selected from the group consisting of Ala, Gly, Ser and Thr.

13. A modular composition comprising:
a) one or more PSPF peptides of claim 1, which may be the same or different;
b) a single stranded or double stranded oligonucleotide;
c) optionally one or more linkers, which may be the same or different;
d) optionally one or more targeting ligands, which may be the same or different;
e) optionally one or more other peptides; and
f) optionally one or more lipids, which may be the same or different.

14. A modular composition comprising:
a) one or more PSPF peptides of claim 1, which may be the same or different;
b) a single stranded or double stranded oligonucleotide; and
c) one or more linkers, which may be the same or different.

15. The modular composition of claim 14, wherein the oligonucleotide is a double stranded siRNA; and wherein each PSPF peptide is independently selected from peptides of SEQ. ID No. 1-24.

16. The modular composition of claim 14, further comprising:
d) one or more ligands, which may be the same or different.

17. The modular composition of claim 16, wherein each ligand is independently selected from the group consisting of D-galactose, N-acetyl-D-galactosamine (GalNAc), GalNAc2, and GalNAc3, GalNAc4, cholesterol, folate, and derivatives thereof.

18. The modular composition of claim 16 comprising:
a) 1-4 PSPF peptides independently selected from SEQ ID No. 1-24;
b) a double stranded siRNA;
c) 1-4 linkers independently selected from Table 4, which may be the same or different; and
d) 1-4 GalNAc ligands, which may be the same or different;
wherein the GalNAc ligands and/or the peptides are attached to the siRNA optionally via linkers.

19. A pharmaceutical composition comprising the PSPF peptide of claim 1 and a pharmaceutically acceptable excipient.

20. A pharmaceutical composition comprising the modular composition of claim 9 and a pharmaceutically acceptable excipient.


SEQUENCE LISTING (24 sequences)

SEQ ID NO: 1 - protein, 28 aa, Artificial Sequence
  “Peptide”
WSDLAQALSDLAQALSDLAQALSDLAQA

SEQ ID NO: 2 - protein, 28 aa, Artificial Sequence
  “Peptide”
WSDLAQGLSDLAQGLSDLAQGLSDLAQG

SEQ ID NO: 3 - protein, 28 aa, Artificial Sequence
  “Peptide”
WSDLAKALSDLAKALSDLAKALSDLAKA

SEQ ID NO: 4 - protein, 28 aa, Artificial Sequence
  “Peptide”
WSDLAKGLSDLAKGLSDLAKGLSDLAKG

SEQ ID NO: 5 - protein, 28 aa, Artificial Sequence
  “Peptide”
WSELAQALSELAQALSELAQALSELAQA

SEQ ID NO: 6 - protein, 28 aa, Artificial Sequence
  “Peptide”
WSELAQGLSELAQGLSELAQGLSELAQG

SEQ ID NO: 7 - protein, 28 aa, Artificial Sequence
  “Peptide”
WSELAKALSELAKALSELAKALSELAKA

SEQ ID NO: 8 - protein, 28 aa, Artificial Sequence
  “Peptide”
WSELAKGLSELAKGLSELAKGLSELAKG

SEQ ID NO: 9 - protein, 28 aa, Artificial Sequence
  “Peptide”
WSHLAQALSHLAQALSHLAQALSHLAQA

SEQ ID NO: 10 - protein, 28 aa, Artificial Sequence
  “Peptide”
WSHLAQGLSHLAQGLSHLAQGLSHLAQG

SEQ ID NO: 11 - protein, 28 aa, Artificial Sequence
  “Peptide”
WSHLAKALSHLAKALSHLAKALSHLAKA

SEQ ID NO: 12 - protein, 28 aa, Artificial Sequence
  “Peptide”
WSHLAKGLSHLAKGLSHLAKGLSHLAKG

SEQ ID NO: 13 - protein, 31 aa, Artificial Sequence
  “Peptide”
WSDLAQALSDLAQALSDLAQALSDLAQAGGC

SEQ ID NO: 14 - protein, 31 aa, Artificial Sequence
  “Peptide”
WSDLAQGLSDLAQGLSDLAQGLSDLAQGGGC

SEQ ID NO: 15 - protein, 31 aa, Artificial Sequence
  “Peptide”
WSDLAKALSDLAKALSDLAKALSDLAKAGGC

SEQ ID NO: 16 - protein, 31 aa, Artificial Sequence
  “Peptide”
WSDLAKGLSDLAKGLSDLAKGLSDLAKGGGC

SEQ ID NO: 17 - protein, 31 aa, Artificial Sequence
  “Peptide”
WSELAQALSELAQALSELAQALSELAQAGGC

SEQ ID NO: 18 - protein, 31 aa, Artificial Sequence
  “Peptide”
WSELAQGLSELAQGLSELAQGLSELAQGGGC

SEQ ID NO: 19 - protein, 31 aa, Artificial Sequence
  “Peptide”
WSELAKALSELAKALSELAKALSELAKAGGC

SEQ ID NO: 20 - protein, 31 aa, Artificial Sequence
  “Peptide”
WSELAKGLSELAKGLSELAKGLSELAKGGGC

SEQ ID NO: 21 - protein, 31 aa, Artificial Sequence
  “Peptide”
WSHLAQALSHLAQALSHLAQALSHLAQAGGC

SEQ ID NO: 22 - protein, 31 aa, Artificial Sequence
  “Peptide”
WSHLAQGLSHLAQGLSHLAQGLSHLAQGGGC

SEQ ID NO: 23 - protein, 31 aa, Artificial Sequence
  “Peptide”
WSHLAKALSHLAKALSHLAKALSHLAKAGGC

SEQ ID NO: 24 - protein, 31 aa, Artificial Sequence
  “Peptide”
WSHLAKGLSHLAKGLSHLAKGLSHLAKGGGC